(19) United States
(12) Patent Application Publication
(10) Pub. No.: US 2020/0368234 A1
(43) Pub. Date: Nov. 26, 2020

(54) PHOTORECEPTOR GENE MODULATOR PHOTOREGULIN 3 FOR TREATMENT OF RETINAL DISEASE

(71) Applicants: University of Washington, Seattle, WA (US); THE J. DAVID GLADSTONE INSTITUTES, San Francisco, CA (US)

(72) Inventors: Thomas A. Reh, Seattle, WA (US); Paul Nakamura, Seattle, WA (US); Andy Shimchuk, Seattle, WA (US); Shibing Tang, Guangzhou (CN); Sheng Ding, Orinda, CA (US)

(73) Assignees: University of Washington, Seattle, WA (US); The J. David Gladstone Institutes, a Testamentary Trust Established Under the Will of J David Gladst, San Francisco, CA (US)

(21) Appl. No.: 16/637,174

(22) PCT Filed: Aug. 10, 2018
(86) PCT No.: PCT/US18/46272

§ 371 Date: Feb. 6, 2020

Related U.S. Application Data
(60) Provisional application No. 62/543,782, filed on Aug. 10, 2017.

(51) Int. Cl. A61K 31/517; A61K 9/00

(52) U.S. Cl. CPC A61K 31/517; A61K 9/0048

(57) ABSTRACT

Methods for decreasing rod gene expression in a retina, methods for decreasing the protein products expressed by rod genes in a retina, methods for treating a disease or condition treatable by decreasing rod gene expression or their protein products in a retina, and methods for treating a retinal disease in a subject using Photoregulin3 (PR3).

### CROSS-REFERENCES TO RELATED APPLICATION

This application claims the benefit of U.S. Application No. 62/543,782, filed Aug. 10, 2017, expressly incorporated herein by reference in its entirety.

### STATEMENT OF GOVERNMENT LICENSE RIGHTS

This invention was made with Government support under Grant Nos. P01 GM081619, R01 EY021374, and R01 EY021482 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 67005_ST25.txt. The text file is 2 KB; was created on Aug. 10, 2018; and is being submitted via EFS-Web with the filing of the specification.

### BACKGROUND OF THE INVENTION

Retinitis pigmentosa (RP) is an inherited retinal degenerative disease with a prevalence of 1 to 3,000-5,000 births. More than 3,000 mutations in about 60 genes have been identified to be associated with RP. Most of these mutations are in genes essential for rod photoreceptor development and function. There is currently no effective medical therapy that slows or prevents rod degeneration in these individuals.

One emerging approach to treating retinal degeneration is through targeting the factors that regulate rod gene expression. Studies of retinal development have identified several transcription factors that regulate photoreceptor gene expression. For example, loss of function mutations in the rod-specific transcription factors Nrl or Nr2e3 cause rods to acquire a more cone-like identity. Conditional knockout has shown that Nrl, is necessary even in mature rods to maintain their normal levels of gene expression. Moreover, the reductions in rod gene expression from deletion of Nrl, with either conditional deletion or CRISPR-Cas9 viral deletion, were sufficient to promote the survival of rods in RP models.

Recently it has been reported that this complex can be modulated using small molecule modulators of Nr2e3 that known as Photoregulins. Treatment of developing or mature retina with Photoregulin1 (PR1) reduces rod gene expression and increases the expression of some cone genes, much like the genetic loss of function mutation in Nr2e3. In addition, treatment of two mouse models of RP, the Rho^{P23H} and the Pde6b^{Rd1} mutations with PR1 slows rod degeneration in vitro. However, in vivo analyses of PR1 were limited by the compounds' potency, solubility, and stability in vivo.

Despite the advances in the development of PR1 compounds, a need exists for compounds that act through Nr2e3, but is more amenable for in vivo studies. The present invention seeks to fulfill this need and provides further related advantages.

### SUMMARY OF THE INVENTION

In one aspect, the invention provides a method for decreasing rod gene expression in a retina. In certain embodiments, the rod gene is Nrl, Nr2e3, Rho, or Gnat1.

In another aspect, the invention provides a method for decreasing rhodopsin expression in a retina.

In the above methods, the retina is contacted with a compound of formula (I):

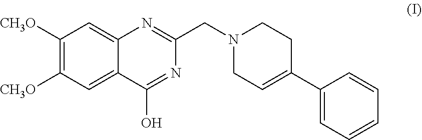

or a pharmaceutically acceptable salt thereof.

In certain embodiments of the above methods, contacting the retina comprises systemic administration or intravitreal injection. In certain embodiments, the retina is a retina of a human subject.

In a further aspect, the invention provides a method for treating a disease or condition treatable by decreasing rod gene expression in a retina.

In yet another aspect, the invention provides a method of treating a retinal disease in a subject.

In the above methods, a therapeutically effective amount of a compound of formula (I):

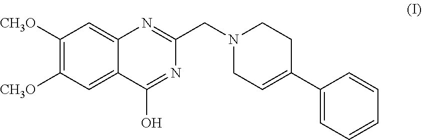

or a pharmaceutically acceptable salt thereof is administered to a subject in need thereof.

In certain embodiments of the above methods, the disease or condition or retinal disease is retinitis pigmentosa, retinal degeneration, macular degeneration, age-related macular degeneration, Stargardt's macular dystrophy, retinal dystrophy, Sorsby's fundus dystrophy, diabetic retinopathy, diabetic maculopathy, retinopathy of prematurity, and ischemia reperfusion related retinal injury. In certain embodiments, the disease or condition or retinal disease is retinitis pigmentosa.

In certain embodiments of the above methods, administering the compound comprises systemic administration or intravitreal injection. In certain embodiments, the subject is a human.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A illustrates the chemical structure of Photoregulin3 (PR3).

FIG. 1B compares dose-response relationship of PR1 and PR3 on Rhodopsin mRNA expression in dissociated retinal cell cultures.

FIG. 1C compares dose-response relationship of PR1 and PR3 on Rhodopsin protein expression in dissociated retinal cell cultures.

FIG. 1D shows intact retinas from P11 mice were explanted in media containing DMSO or 0.3 μM PR3 for 3 DIV and then stained for S Opsin (green) and DAPI in a whole-mount preparation. Scale bar represents 50 μm. E. PR3-treated retinas had more S Opsin+ cells per 100 μm×100 μm field in the ventral retina compared to DMSO-treated retinas (n=3, *p<0.05, Student's t-test).

FIG. 1E is a graph quantifying the effects of PR3 on S-opsin expression; treatment conditions were the same as those in FIG. 1D. S-opsin+ cones are predominantly present in the ventral retina of mice. PR3-treated retinas had significantly more S Opsin+ cells per 100 μm×100 μm field in the ventral retina compared to DMSO-treated retinas (n=3, *p<0.05, Student's t-test).

FIG. 1F illustrates an isothermal titration calorimetric (ITC) study of PR3 binding to Nr2e3. Nr2e3 protein was expressed as a fusion protein from the expression vector pVP16. The fusion protein was incubated with TEV overnight at 4° C. and then the His8-MBP tags were separated from Nr2e3 by ion exchange chromatography. For isothermal titration calorimetry, 100 mM PR3 was injected into 20 mM Nr2e3 in a MicroCal ITC-200 (Malvern). ITC qualitatively showed a direct interaction between PR3 and Nr2e3, with an estimated K_{d} of 67 μM using a one site model.

FIG. 2A shows RNA sequencing results plotting log Fold Change (logFC) against log RPKM of wild type mice treated with DMSO vehicle or 10 mg/kg PR3 shows robust reduction in rod photoreceptor genes. (n=2 mice per condition)

FIG. 2B shows gene ontology analysis (http://geneontology.org/page/go-enrichment-analysis) results for largest changes (top 100) in gene expression assessed by RNA sequencing.

FIG. 2C compares electron microscope micrographs of retinal sections of wild type mice treated with 10 mg/kg PR3 or DMSO vehicle. Compared to controls, PR3 retinas have arrested inner and outer segment development and less compact heterochromatin in the ONL. Scale bars represent 10 μm (top) and 2 μm (bottom).

FIG. 3A illustrates the timeline of photoreceptor degeneration in the Rhodopsin^{P23H} mouse and experimental design.

FIG. 3B compares immunofluorescence staining for Rhodopsin, S Opsin, Otx2, and Cone Arrestin on retinal sections from Rhodopsin^{P23H} mice and demonstrates preservation of photoreceptors with PR3 treatment. Scale bar represents 50 μm.

FIG. 3C compares counts for rows of DAPI+ cells in the central and peripheral ONL and show greater survival of photoreceptors with PR3 treatment (n≥7, *p<0.05, Student's t-test).

FIG. 3D compares qPCR on whole retinas from Rhodopsin^{P23H} mice treated with DMSO or PR3 and shows greater expression of photoreceptor genes Recoverin, Rhodopsin, and S Opsin with PR3 treatment (n≥6, *p<0.05, Student's t-test).

FIG. 4A compares scotopic b-wave amplitudes from P21 Rhodopsin^{P23H} mice treated with 10 mg/kg PR3 or DMSO vehicle (n=4-8, *p<0.05, t-test).

FIG. 4B compares representative scotopic ERG waveforms from a single DMSO vehicle mouse.

FIG. 4C compares representative scotopic ERG waveforms from a single PR3-treated mouse.

FIG. 4D compares photopic b-wave amplitudes from P21 Rhodopsin^{P23H} mice treated with 10 mg/kg PR3 or DMSO vehicle (n=4-8, *p<0.05, t-test).

FIG. 4E compares representative photopic ERG waveforms from a single control (DMSO) mouse.

FIG. 4F compares representative photopic ERG waveforms from a single PR3-treated mouse.

### DETAILED DESCRIPTION OF THE INVENTION

The present invention provides methods for decreasing rod gene expression in a retina, methods for decreasing the protein products (e.g., rhodopsin) expressed by rod genes in a retina, methods for treating a disease or condition treatable by decreasing rod gene expression or their protein products in a retina, and methods for treating a retinal disease in a subject. In the methods, the retina or subject are treated with Photoregulin3 (PR3) to achieve the advantageous result of decreasing rod gene expression, thereby decreasing the expression of their protein products, and consequently treating a disease or condition treatable by decreasing rod gene expression or their protein products. As described herein, the results demonstrate that PR3 is effective for treating retinal diseases such as retinitis pigmentosa (RP).

Decreasing Rod Gene Expression and their Protein Products

In one aspect, the invention provides a method for decreasing rod gene expression in a retina.

In one embodiment, the method includes contacting a retina with PR3, a compound of formula (I):

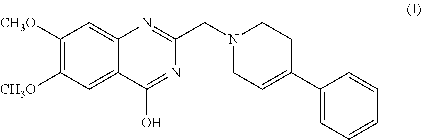

or a pharmaceutically acceptable salt thereof.

Rod genes whose expression are effectively reduced in the practice of the methods of the invention include Nrl, Nr2e3, Rho, Gnat1, and Pde6b.

In certain embodiments of the above methods, contacting the retina comprises systemic administration of the compound to the subject or intravitreal injection of the compound.

In another aspect, the invention provides a method for reducing the expression of protein products derived from rod genes. In certain embodiments, the invention provides a method for decreasing rhodopsin expression in a retina.

In one embodiment of this method, a retina is treated with a compound of formula (I) or a pharmaceutically acceptable salt thereof, as described above.

In certain embodiments of the above methods, treating the retina comprises systemic administration of the compound to the subject or intravitreal injection of the compound.

Treating Diseases or Conditions Treatable by Decreasing Rod Gene Expression or their Protein Products

In a further aspect of the invention, methods are provided for treating a disease or condition treatable by decreasing rod gene expression, or their protein products, in a retina.

In certain embodiments, the methods include administering to a subject in need thereof a therapeutically effective amount of a compound of formula (I) or a pharmaceutically acceptable salt thereof, as described above.

Representative diseases or conditions treatable by decreasing rod gene expression, or their protein products, in a retina include retinitis pigmentosa, retinal degeneration, macular degeneration, age-related macular degeneration, Stargardt's macular dystrophy, retinal dystrophy, Sorsby's fundus dystrophy, diabetic retinopathy, diabetic maculopathy, retinopathy of prematurity, and ischemia reperfusion related retinal injury. In one embodiment, the treatable disease or condition is retinitis pigmentosa.

In certain embodiments of the above methods, administering the compound comprises systemic administration of the compound to the subject or intravitreal injection of the compound.

Treating Retinal Disease

In another aspect, the invention provides methods for treating a retinal disease in a subject.

In certain embodiments, the methods include administering to a subject in need thereof a therapeutically effective amount of a compound having formula (I) or a pharmaceutically acceptable salt thereof, as described above.

Representative retinal diseases treatable by the methods of the invention include retinitis pigmentosa, retinal degeneration, macular degeneration, age-related macular degeneration, Stargardt's macular dystrophy, retinal dystrophy, Sorsby's fundus dystrophy, diabetic retinopathy, diabetic maculopathy, retinopathy of prematurity, and ischemia reperfusion related retinal injury. In one embodiment, the treatable disease or condition is retinitis pigmentosa.

In certain embodiments of the above methods, administering the compound comprises systemic administration of the compound to the subject or intravitreal injection of the compound.

In the methods of the invention that are methods of treatment, the term “therapeutically effective amount” refers to an amount effective, at dosages and for periods of time necessary, to achieve the desired therapeutic result, such as reduced levels of rod gene expression or their protein products. A therapeutically effective amount of a compound may vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the administered compound are outweighed by the therapeutically beneficial effects.

It is to be noted that dosage values can vary with the severity of the condition to be alleviated. For any particular subject, specific dosage regimens can be adjusted over time according to the individual need and the professional judgment of the person administering or supervising the administration of the compositions. Dosage ranges set forth herein are exemplary only and do not limit the dosage ranges that can be selected by a medical practitioner. The amount of active compound in the composition can vary according to factors such as the disease state, age, sex, and weight of the subject. Dosage regimens can be adjusted to provide the optimum therapeutic response. For example, a single bolus can be administered, several divided doses can be administered over time or the dose can be proportionally reduced or increased as indicated by the exigencies of the therapeutic situation.

In the methods, the administration of the compound can be a local administration (e.g., administration to the eye), or systemic administration to the subject. The term “subject” is intended to include mammalian organisms. Examples of subjects include humans and non-human mammals. In specific embodiments of the invention, the subject is a human.

The terms “administering,” “contacting,” or “treating” include any method of delivery of a compounds or a pharmaceutical composition comprising the compound into a subject's system or to a particular region of the subject (e.g., eye).

Increasing Cone Gene Expression Methods

In further aspects, the invention provides methods for increasing cone gene expression, or their protein products, in a retina.

In one embodiment of this method, a retina is treated or contacted with a compound having formula (I) or a pharmaceutically acceptable salt thereof, as described above.

In certain embodiments of the above methods, treating or contacting the retina comprises systemic administration of the compound to the subject or intravitreal injection of the compound.

In certain embodiments of the above methods, treating or contacting the retina comprises systemic administration of the compound to the subject or intravitreal injection of the compound.

Pharmaceutical Compositions

In another aspect, the invention provides a pharmaceutical composition that includes a pharmaceutically acceptable carrier and a compound of formula (I) or a pharmaceutically acceptable salt thereof.

Suitable carriers include those suitable for administration to an animal (e.g., a human subject). Pharmaceutical compositions suitable for injectable use include sterile aqueous solutions (e.g., saline, dextrose) and dispersions.

The compositions of the invention can be orally administered, for example, with an inert diluent or carrier, enclosed in hard or soft shell gelatin capsule, or compressed into tablets. For oral therapeutic administration, the compounds and compositions can be combined with excipients and used in the form of ingestible tablets, buccal tables, troches, capsules, elixirs, suspensions, syrups, wafers, and the like. The amount of active compounds in such therapeutically useful compositions is such that a suitable dosage is obtained.

The compositions of the invention can be administered parenterally. Solutions of the compounds as free base or pharmacologically acceptable salts can be prepared in water suitably mixed with additives, such as surfactants. Dispersions can also be prepared in in oils.

Methods for Using Photoregulin 3 (PR3)

The following description relates to Photoregulin3 or PR3 (a small molecule antagonist of Nr2e3a) useful in the methods of the invention.

Nr2e3 is a retina-specific nuclear receptor and a key regulator of photoreceptor gene expression. Regulation of rod gene expression has emerged as a potential therapeutic strategy to treat retinal degenerative diseases like Retinitis Pigmentosa (RP). Photoregulin1 (PR1) is a small molecule modulator of Nr2e3 that regulates the expression of photoreceptor-specific genes. Manipulation of photoreceptor gene expression with PR1 slows the progression of retinal degeneration in vitro in the Rho^{P23H} and Pde6b^{Rd1} models of RP. However, in vivo analyses with this series were limited by the compounds' potency, solubility, and stability in vivo. As described herein, a structurally unrelated compound, Photoregulin3 (PR3), has been identified that is more potent than the compounds of the PR1 series. After systemic delivery, PR3 has large effects on rod photoreceptor gene expression (e.g. Nrl, Rhodopsin, Gnat1) by RNA sequencing analysis. To determine the effectiveness of PR3 as a potential therapy for RP, Rho^{P23H} mice were treated with PR3 or vehicle from postnatal day 12-14 (P12-14) until P20 and assessed retinal structure and function at P21. PR3-treated Rho^{P23H} mice had larger scotopic and photopic ERG responses than littermate controls, in addition to significantly decreased degeneration of photoreceptors. Together, the data suggest that pharmacological disruption of Nr2e3 signaling may be a therapeutically advantageous strategy for the treatment of RP and other degenerative diseases of the retina.

The PR3 treatment described herein demonstrates anatomical and functional preservation of the retina in Rho^{P23H} mice, providing proof-of-concept of this therapeutic strategy for the treatment of RP.

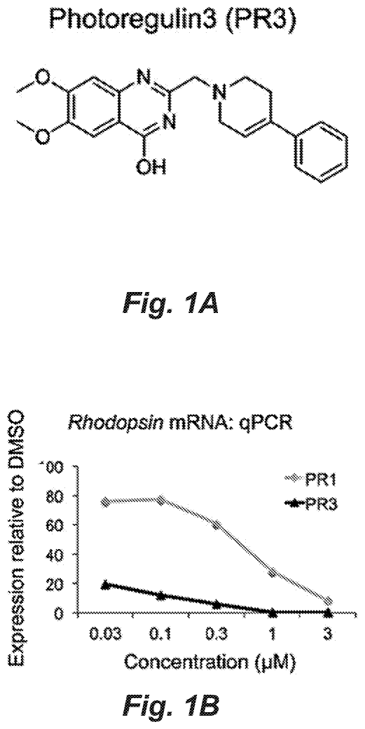

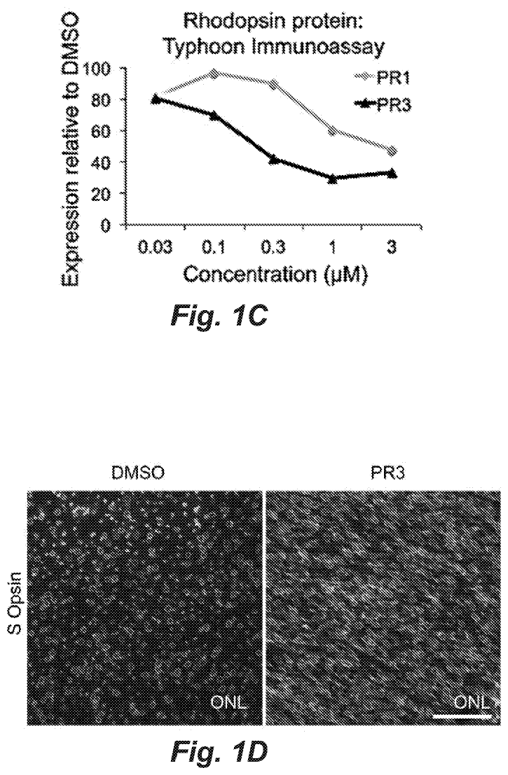

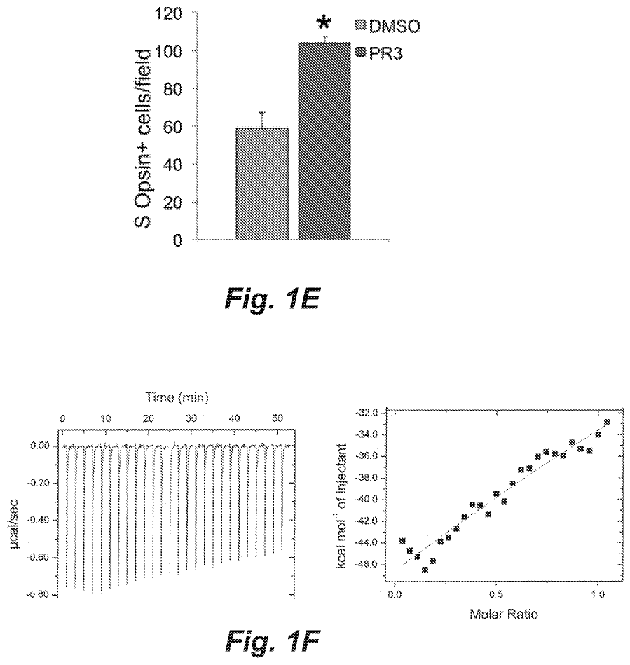

In order to identify compounds that may target Nr2e3 a cheminformatic strategy was used to screen hit compounds previously identified to interact with recombinant Nr2e3 in transfected CHO-S cells in a luciferase-based assay (PubChem Assay IDs: 602229, 624378, 624394, and 651849). As a secondary screen for initial hits, dissociated primary retinal cell cultures were used and assayed Rhodopsin, because it is a well-defined target of Nr2e3 signaling and is expressed at high levels exclusively in rod photoreceptors. Retina were dissociated from postnatal day 5 (P5) mice and cultured them in media containing the small molecules. After treatment for 2 days, the cells were collected for qPCR analysis and quantified Rhodopsin expression. One compound, Photoregulin3 (PR3; FIG. 1A), showed robust reduction in Rhodopsin compared to DMSO and PR1 treatment at all concentrations (FIG. 1). This finding was confirmed with an immunofluorescence assay to examine Rhodopsin protein expression in dissociated retinal cultures from P5 mice. Similar to the qPCR results, treatment with PR3 resulted in reduced expression of Rhodopsin compared to DMSO vehicle and PR1 (FIG. 1C).

Mutations in Nr2e3 result in an increased number of S Opsin+ photoreceptors as well as a reduction in rod gene expression. To determine if PR3 treatment also affects cone gene expression, intact retinas were explanted from P11 wild type mice in media containing DMSO or PR3 for 3 days. Intact retinas were used for this experiment to assess changes in dorsal and ventral retina independently. After fixation and whole mount immunostaining, S Opsin+ cells were counted in the ventral retina. Similar to Nr2e3 mutations, treatment with PR3 resulted in an increase in the number S Opsin+ cells in the ventral retina (FIGS. 1D-1E).

PR3 was initially identified as a chemical modulator of Nr2e3 in a luciferase-based assay that identified ligands by disruption of the Nr2e3-NCOR dimer complex (PubChem Assay IDs: 602229, 624378, 624394, and 651849). To confirm a direct Nr2e3-PR3 interaction, isothermal titration calorimetry (ITC) was used. Consistent with other assays, ITC qualitatively showed a direct interaction between PR3 and Nr2e3 (estimated K_{d} of 67 μM using one site model; FIG. 1F).

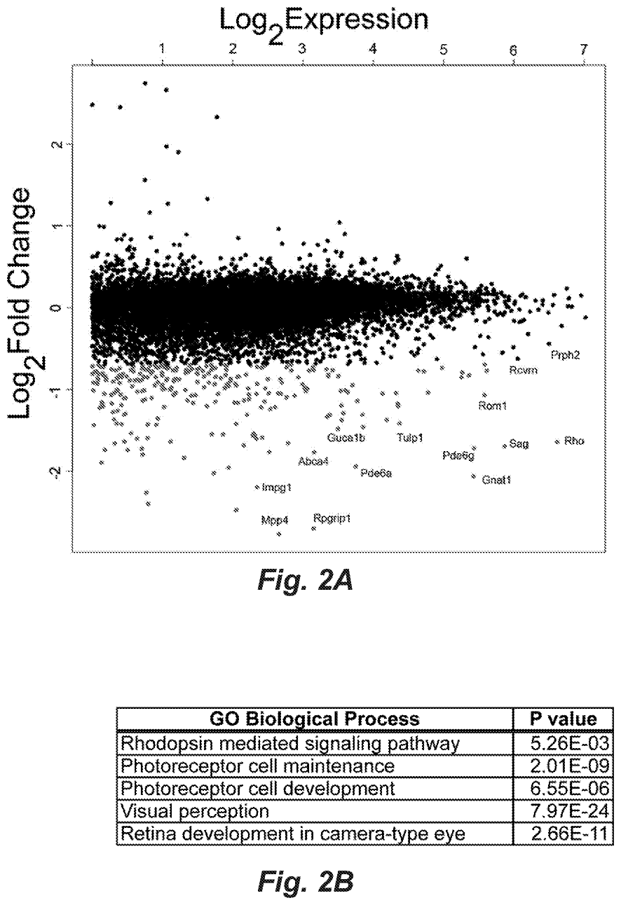

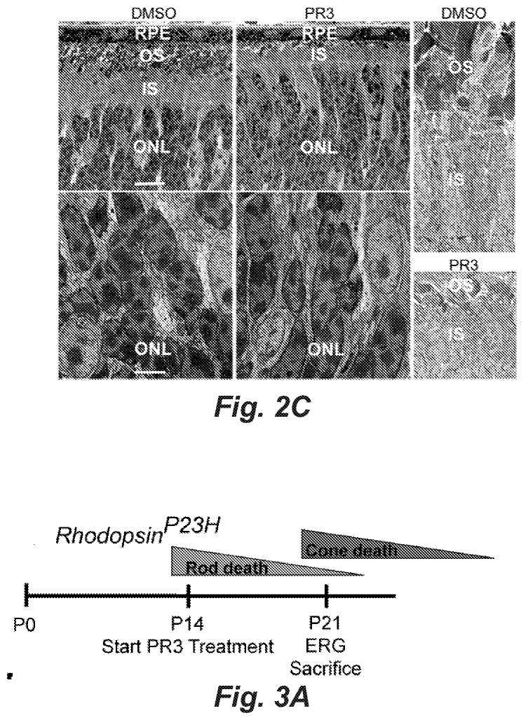

Nr2e3 signaling is important for rod photoreceptor cell fate, development and maturation, and maintenance of expression. To determine the effect of PR3 treatment on gene expression in postmitotic retinal cells, wild type mice were systemically treated (intraperitoneal injection) with PR3 or vehicle at P12. At P13, 24 h after the injection, the retinas were collected for global transcriptome analysis by RNA sequencing. A decrease in most rod photoreceptor-specific transcripts was observed (FIGS. 2A and 2B). Similar to conditional knockout of Nrl and knockdown of Nrl by CRISPR/Cas9 in postmitotic photoreceptors, large and global increases in cone gene expression were not observed. However, genes expressed in both rod and cone photoreceptors, like Crx and Otx2, that are upstream of Nr2e3 showed no difference in expression between control and PR3 treatment. In addition, changes in genes expressed in other retinal cell types were not observed, indicating specificity of the PR3 for photoreceptors, and increases in cell death genes or cell stress genes were not observed, indicating that the compound is not toxic to retinal cells.

Electron microscopy (EM) was used to perform an ultrastructural analysis of photoreceptor morphology after PR3 treatment. Wild type mice were intraperitoneally injected with vehicle or PR3 for 3 consecutive days starting at P11. At P14, 24 h after the third injection, the mice were euthanized and processed their retinas for EM. Photoreceptor outer segments begin to form during the second and third postnatal week; genetic loss-of-function mutations in Nr2e3 lead to an impairment in rod outer segment formation. PR3 treatment prevented outer segment development; outer segments of PR3-treated photoreceptors were strikingly truncated compared to controls (FIG. 2C). Any indication of photoreceptor apoptosis induced by PR3 treatment upon examination of outer nuclear layer (ONL) nuclei (FIG. 2C) was not observed, indicating that the effect on rod development was not due to an increase in cell death. Interestingly, rod nuclei of PR3-treated retinas contained smaller patches of densely packed heterochromatin compared to DMSO retinas (FIG. 2C), consistent with the cells adopting a more cone-like identity.

It has been recently shown that reductions in rod gene expression caused by treatment with PR1 were sufficient to slow the degeneration of Rho^{P23H} photoreceptors in vitro. The Rho^{P23H} mutation causes misfolding of Rhodopsin in rod photoreceptors, which leads to activation of the unfolded protein response and eventually results in rod and cone death. In Rho^{P23H} mice, most rod photoreceptors undergo apoptosis by the end of the third postnatal week.

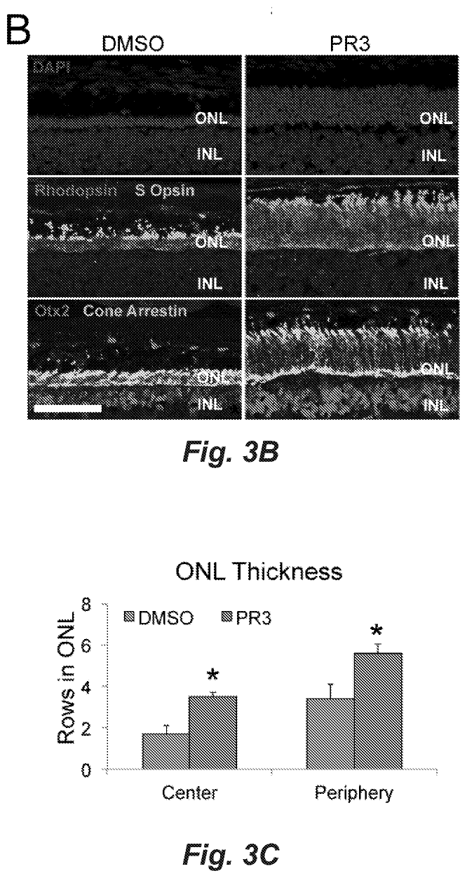

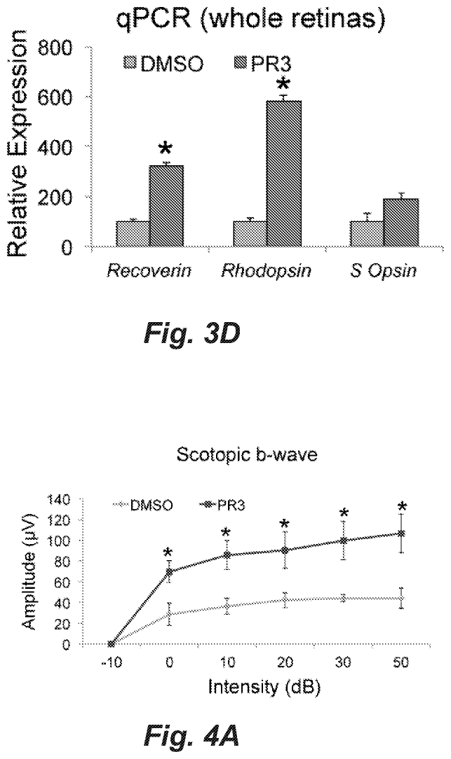

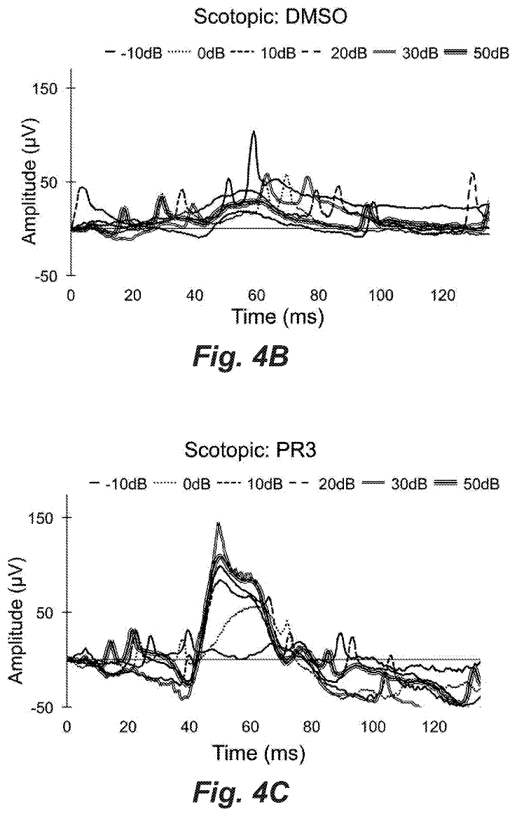

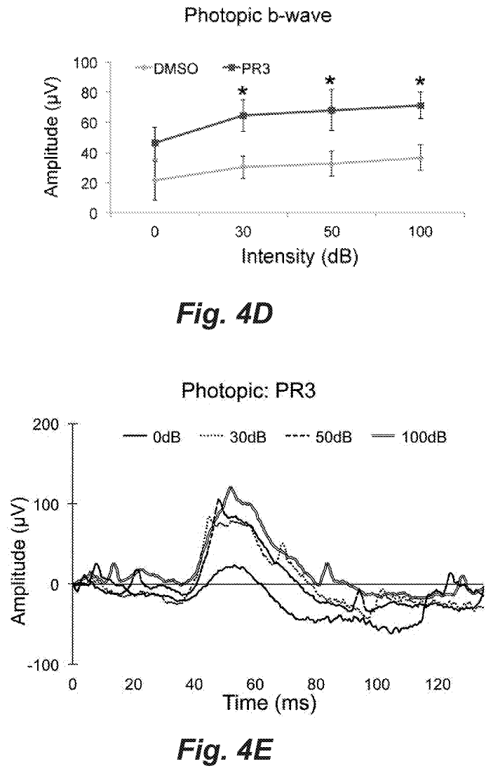

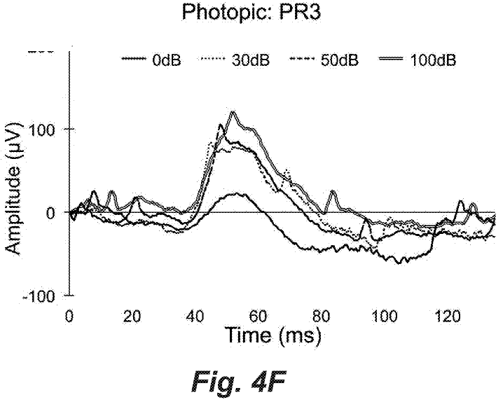

To determine whether photoreceptor degeneration can be prevented, Rho^{P23H} mice were treated with PR3 or vehicle from P12-P14 until P21, during the period of rod photoreceptor death (FIG. 3A). At P21, visual function was assessed with ERGs and euthanized the mice for histological and qPCR analyses. At P21, control Rho^{P23H} mice had only 2-3 rows of photoreceptors remaining in their ONL (FIGS. 3B and 3C). Rods were sparse and there were few remaining cones (S Opsin+ and Cone Arrestin+ photoreceptors). Control Rho^{P23H} mice had minimal scotopic and photopic b-wave amplitudes by ERG analysis (FIGS. 4A, 4B, 4D, and 4E). By contrast, retinas from PR3-treated Rho^{P23H} mice had several rows of rod and cone photoreceptors in the ONL (FIGS. 3B and 3C). The surviving cones in the PR3-treated retinas were more elongated and healthier than in the DMSO control retinas. The histological results with qPCR were confirmed on retinas from control and PR3 Rho^{P23H} mice and it was found that treated mice had more expression of Recoverin and Rhodopsin, indicating greater photoreceptor cell survival (FIG. 3D). ERG analysis of PR3-treated Rho^{P23H} mice showed significantly elevated scotopic and photopic b-wave amplitudes at most stimulation intensities compared to littermate controls (FIGS. 4A, 4C, 4D, and 4F). Together, these data support the conclusion that PR3 treatment prevented structural and functional degeneration of photoreceptors in this model of RP.

As described herein, photoreceptor degeneration in the Rho^{P23H} mouse was prevented, which is the first report of successful treatment of this RP model with a small molecule in vivo. The expression of photoreceptor genes was reduced by targeting the rod-specific nuclear receptor Nr2e3 with a small molecule modulator. Treatment with PR3 decreased rod gene expression, and was sufficient to functionally and structurally preserve photoreceptors in the Rho^{P23H} mouse. Previous studies have shown that genetic manipulation of the rod photoreceptor differentiation pathway may useful for the treatment of multiple RP models. Conditional deletion of Nrl in adult mouse rods prevents degenerations in the Rho^{-/-} model of recessive RP. More recently, knockdown of Nrl by AAV-CRISPR/Cas9 gave long-term histological and functional preservation of photoreceptors in three different RP models, the Rho^{-/-}, the Pde6b^{Rd10}, and the Rho^{P34S}. The results described herein show that a small molecule targeting this same complex is also effective at slowing rod degeneration, in a particularly aggressive RP model and provides a novel pathway for medical therapy of retinal degeneration.

Materials and Methods

Mice

C57Bl/6 (Jackson Stock No: 000664) and Rho^{P23H} (Jackson Stock No: 017628) were used at the indicated ages. All mice were housed by the Department of Comparative Medicine at the University of Washington and protocols were approved by the University of Washington Institutional Animal Care and Use Committee. The research was carried out in accordance with the ARVO statement for the Use of Animals in Ophthalmic and Vision Research.

Photoregulin3

Photoregulin3 was identified by searching previous small molecule screens with SciFinder and PubChem for Nr2e3 interacting molecules. It was initially obtained from ChemDiv and then synthesized and purified in large quantities in the lab after initial screening. For in vivo experiments, mice were injected intraperitoneally with PR3 dissolved in DMSO at 10 mg/kg.

Dissociated Retinal Cultures

Retinas were dissected from postnatal day 5 (P5) mice and dissociated by treatment with 0.5% Trypsin diluted in calcium- and magnesium-free HBSS from 10 minutes at 37° C. Trypsin was inactivated by adding an equal volume of FBS and cells were pelleted by centrifugation at 4° C. and resuspended in media (Neurobasal-A containing 1% FBS, 1% N₂, 1% B27, 1% Pen/Strep, and 0.5% L-glutamine). For qPCR, cells were plated into 24-well tissue culture plates at a density of 1 retina/well (see qPCR section below). For the immunofluorescence assay, cells were plated into 96-well black walled, clear bottom tissue culture plates at a density of 1 retina/5 wells. Small molecules were diluted in media and were added the day following dissociation. After two days of treatment, cells were fixed with 4% PFA for 20 minutes at room temperature, blocked with blocking solution (10% Normal Horse Serum and 0.5% Triton X-100 diluted in 1×PBS) for 1 hour at room temperature, and incubated overnight at 4° C. with primary antibodies generated against Rhodopsin (1:250; Rho4D2, Robert Molday, University of British Columbia) diluted in blocking solution. The following day, wells were washed with 1×PBS and then incubated with species appropriate, fluorescently labeled secondary antibodies diluted in blocking solution for 1 hour at room temperature. Wells were washed three times, counterstained with ToPro3, and the entire plate was imaged using a GE Typhoon FLA 9400 imager. Optical density measurements were obtained from the plate scans using ImageJ software and Rhodopsin expression was normalized to ToPro3 nuclear stain.

Quantitative Real-Time PCR

RNA from retinas was isolated using TRIzol (Invitrogen) and cDNA was synthesized using the iScript cDNA synthesis kit (Bio-Rad). SSO Fast (Bio-Rad) was used for quantitative real-time PCR. For analysis, values were normalized to Gapdh (ΔCt) and ΔΔCt between DMSO and compound-treated samples was expressed as percent of DMSO treated controls (100*2\hat{ }ΔΔCt). Student's t-tests were performed on ΔCt values. The following primer sequences were used: Gapdh (F: GGCATTGCTCTCAATGACAA (SEQ ID NO: 1), R: CTTGCTCAGTGTCCTTGCTG (SEQ ID NO: 2)), Rhodopsin (F: CCCTTCTCCAACGTCACAGG (SEQ ID NO: 3), R: TGAGGAAGTTGATGGGGAAGC (SEQ ID NO: 4)), Opn1sw (F: CAGCATCCGCTTCAACTCCAA (SEQ ID NO: 5), R: GCAGATGAGGGAAAGAGGAATGA (SEQ ID NO: 6)), Recoverin (F: ACGACGTAGACGGCAATGG (SEQ ID NO: 7), R: CCGCTTTTCTGGGGTGTTTT (SEQ ID NO: 8)).

Retinal Explant Cultures

Intact retinas without RPE from P11 C57Bl/6 mice were explanted on 0.4 μm pore tissue culture inserts in media (Neurobasal-A containing 1% FBS, 1% N₂, 1% B27, 1% Pen/Strep, and 0.5% L-Glutamine) containing DMSO or 0.3 μM Photoregulin3. Full media changes were performed every other day. Explants were fixed with 4% PFA for 20 minutes at room temperature, blocked with blocking solution (10% Normal Horse Serum and 0.5% Triton X-100 diluted in 1×PBS) for 1 hour at room temperature, and incubated overnight at 4° C. with primary antibodies generated against S Opsin (1:400, SCBT, sc-14363). The following day, the explants were washed with 1×PBS, and then incubated with a species appropriate, fluorescently-labeled secondary antibody diluted in blocking solution overnight, followed by washing with 1×PBS and DAPI staining. The explants were transferred to slides and coverslipped with Fluoromount-G (SouthernBiotech). An Olympus FluoView FV1000 was used for confocal microscopy. Cells were counted from single plane confocal images taken at fixed settings.

Immunofluorescence

Eyecups were fixed in 4% PFA in 1×PBS for 20 minutes at room temperature and then cryoprotected in 30% sucrose in 1×PBS overnight at 4° C. Samples were embedded in OCT (Sakura Finetek), frozen on dry ice, and then sectioned at 16-18 μm on a cryostat (Leica). Slides were blocked with a solution containing 10% Normal Horse Serum and 0.5% Triton X-100 in 1×PBS for 1 hour at room temperature and then stained overnight at 4° C. with primary antibodies (Rho4D2 at 1:250 from Robert Molday (University of British Columbia), S Opsin at 1:400 from SCBT: sc-14363, Cone Arrestin at 1:1,000 from Millipore: AB15282, Otx2 at 1:200 from R&D Systems: BAF1979) diluted in blocking solution. Slides were washed three times with 1×PBS the following day and then incubated in fluorescently labeled secondary antibodies diluted in blocking solution for 2 hours at room temperature, stained with DAPI, washed, and coverslipped using Fluoromount-G (SouthernBiotech). An Olympus FluoView FV1000 was used for confocal microscopy. Cells were counted from single plane confocal images taken at fixed settings. Counts in the central retina were taken adjacent to the optic nerve head (50 μm from the nerve head on the ventral side) and counts in the peripheral retina were taken 50 μm from the peripheral edge on the ventral side.

Isothermal Titration Calorimetry

Nr2e3 protein (aa 90-410) was expressed as a His8-MBP-TEV fusion protein from the expression vector pVP16 (DNASU Plasmid ID: HsCD00084154). E. coli BL21 (DE3) cells were grown to an OD600 of 1, and then induced with 0.2 mM IPTG at 16° C. overnight. Cells were harvested, resuspended in extract buffer (20 mM Tris pH 8, 200 mM NaCl, 10% glycerol, 5 mM 2-mercaptoethanol, and saturated PMSF diluted 1:1,000), and then lysed by sonication on ice. Lysates were centrifuged at 4° C. and the supernatant was loaded onto an equilibrated column containing 5 mL of Ni-NTA agarose (Qiagen). The column was washed with 20 mM Tris pH 8, 1M NaCl, 5 mM 2-mercaptoethanol, and 40 mM imidazole, and then the protein was eluted with 20 mM Tris pH 8, 200 mM NaCl, 5 mM 2-mercaptoethanol, and 100 mM imidazole. The fusion protein was incubated with TEV overnight at 4° C. and then the His8-MBP tags were separated from NR2E3 by ion exchange chromatography. For isothermal titration calorimetry, 100 μM PR3 was injected into 20 μM Nr2e3 in 10 mM sodium phosphate buffer pH 8 with 50 mM NaCl and 0.5% DMSO in a MicroCal ITC-200 (Malvern) and the data was analyzed with Origin 7.0 software.

RNA Sequencing

RNA from retinas was isolated using TRIzol (Invitrogen) and total RNA integrity was checking using an Agilent 4200 TapeStation and quantified using a Trinean DropSense96 spectrophotometer. RNA-seq libraries were prepared from total RNA using the TruSeq RNA Sample Prep kit (Illumina) and a Sciclone NGSx Workstation (PerkinElmer). Library size distributions were validated using an Agilent 4200 TapeStation. Additional Library quality control, blending of pooled indexed libraries, and cluster optimization were performed using Life Technologies' Invitrogen Qubit Fluorometer. RNA-seq libraries were pooled (4-plex) and clustered onto a flow cell lane. Sequencing was performed using an Illumina HiSeq 2500 in rapid mode employing a paired-end, 50 base read length (PE50) sequencing strategy.

Electron Microscopy

Mice were euthanized by CO₂, and then perfused with 0.9% saline followed by 4% glutaraldehyde in 0.1 M sodium cacodylate buffer. Eye cups were fixed in 4% glutaraldehyde in 0.1 M sodium cacodylate buffer, washed with 0.1 M sodium cacodylate buffer, and then post-fixed in 2% osmium tetroxide. After fixation, eye cups were washed with water, dehydrated through a graded series of ethanol, incubated in propylene oxide and then epon araidite, polymerized overnight at 60° C., and then sectioned at a thickness of 70 nm. Images were obtained using a JEOL JEM-1230 electron microscope.

ERGs

Mice were dark adapted overnight (12-18 hours). All subsequent steps were carried out under dim red light. Mice were placed in an anesthesia chamber and anesthetized with 1.5-3% isoflurane gas. Mice were transferred from the anesthesia chamber to a heated platform maintained at 37° C. and positioned in a nose cone to maintain a constant flow of isoflurane. Drops of 1% tropicamide and 2.5% phenylephrine hydrochloride were applied to each eye. A reference needle electrode was placed subdermally on the top of the head and a ground needle electrode was placed subdermally in the tail. Drops of 1.5% methyl cellulose were applied to each eye and contact lens electrodes were placed over each eye.

Dim red light was turned off and the platform was positioned inside of an LKC Technologies UTAS BigShot ganzfeld and a series of flashes of increasing intensity were delivered scotopically. A series of photopic flashes were performed immediately following the series of scotopic flashes.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for decreasing rod gene expression in a retina, comprising contacting a retina with a compound of formula (I): 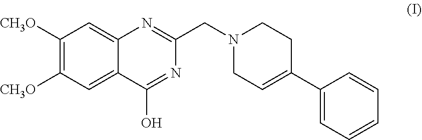
or a pharmaceutically acceptable salt thereof.

2. The method of claim 1, wherein the rod gene is selected from the group consisting of Nrl, Nr2e3, Rho, and Gnat1.

3. The method of claim 1, wherein contacting the retina comprises systemic administration or intravitreal injection.

4. The method of claim 1, wherein the retina is a retina of a human subject.

5. A method for treating a disease or condition treatable by decreasing rod gene expression in a retina, comprising administering to a subject in need thereof a therapeutically effective amount of a compound of formula (I): 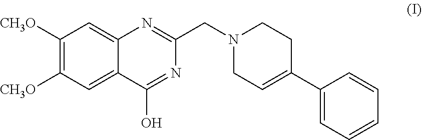
or a pharmaceutically acceptable salt thereof.

6. The method of claim 5, wherein the disease or condition is selected from the group consisting of retinitis pigmentosa, retinal degeneration, macular degeneration, age-related macular degeneration, Stargardt's macular dystrophy, retinal dystrophy, Sorsby's fundus dystrophy, diabetic retinopathy, diabetic maculopathy, retinopathy of prematurity, and ischemia reperfusion related retinal injury.

7. The method of claim 5, wherein the retinal disease is retinitis pigmentosa.

8. The method of claim 5, wherein administering the compound comprises systemic administration or intravitreal injection.

9. The method of claim 5, wherein the subject is a human.

10. A method for decreasing rhodopsin expression in a retina, comprising treating a retina with a compound of formula (I): 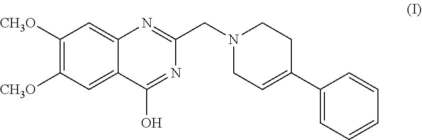
or a pharmaceutically acceptable salt thereof.

11. The method of claim 10, wherein the retina is a retina of a human subject.

12. A method of treating a retinal disease in a subject, comprising administering to a subject in need thereof a therapeutically effective amount of a compound of formula (I): 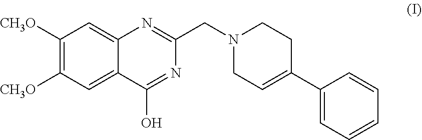
or a pharmaceutically acceptable salt thereof.

13. The method of claim 12, wherein the retinal disease is selected from the group consisting of retinitis pigmentosa, retinal degeneration, macular degeneration, age-related macular degeneration, Stargardt's macular dystrophy, retinal dystrophy, Sorsby's fundus dystrophy, diabetic retinopathy, diabetic maculopathy, retinopathy of prematurity, and ischemia reperfusion related retinal injury.

14. The method of claim 12, wherein the retinal disease is retinitis pigmentosa.

15. The method of claim 12, wherein the subject is a human.

16. The method of claim 12, wherein the administering the compound comprises systemic administration or intravitreal injection.


SEQUENCE LISTING (8 sequences)

SEQ ID NO: 1 - DNA, 20 bp, Artificial Sequence
  primer sequence
ggcattgctctcaatgacaa

SEQ ID NO: 2 - DNA, 20 bp, Artificial Sequence
  primer sequence
cttgctcagtgtccttgctg

SEQ ID NO: 3 - DNA, 20 bp, Artificial Sequence
  primer sequence
cccttctccaacgtcacagg

SEQ ID NO: 4 - DNA, 21 bp, Artificial Sequence
  primer sequence
tgaggaagttgatggggaagc

SEQ ID NO: 5 - DNA, 21 bp, Artificial Sequence
  primer sequence
cagcatccgcttcaactccaa

SEQ ID NO: 6 - DNA, 23 bp, Artificial Sequence
  primer sequence
gcagatgagggaaagaggaatga

SEQ ID NO: 7 - DNA, 19 bp, Artificial Sequence
  primer sequence
acgacgtagacggcaatgg

SEQ ID NO: 8 - DNA, 20 bp, Artificial Sequence
  primer sequence
ccgcttttctggggtgtttt